(19) United States
(12) Patent Application Publication
(10) Pub. No.: US 2006/0009479 A1
(43) Pub. Date: Jan. 12, 2006

(54) Process for the synthesis of hydromorphone

(76) Inventors: Timothy Samuel Bailey, Blackstone Heights (AU); Paul Stanley Gee, Legana (AU); Robert Rezaie, Blackstone Heights (AU)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/178,717

(22) Filed: Jul. 11, 2005

Related U.S. Application Data
(60) Provisional application No. 60/587,304, filed on Jul. 9, 2004.

(51) Int. Cl. C07D 489/02; A61K 31/485

(52) U.S. Cl. 514/282; 546/44

(57) ABSTRACT

There is described a method for converting oripavine to hydromorphone or a physiologically acceptable salt thereof such as hydromorphone hydrochloride involving generation of 8,14-dihydrooripavine utilising diimine.

### FIELD OF THE INVENTION

The present invention relates to a method for converting oripavine to hydromorphone or a physiologically acceptable salt thereof involving the generation of the intermediate 8,14-dihydrooripavine.

### BACKGROUND OF THE INVENTION

Hydromorphone is a synthetic derivative of morphine with an oral analgesic potency about 10 times that of morphine.

A number of processes for the synthesis of hydromorphone utilising morphine as the starting reagent are described in U.S. Pat. No. 2,628,962, U.S. Pat. No. 2,649,454 and U.S. Pat. No. 2,654,756. These processes are based on the Oppenauer oxidation of dihydromorphine with the overall process involving two steps, namely the hydrogenation of morphine to dihydromorphine, then the oxidation of dihydromorphine to hydromorphone. The biotransformation of morphinone to hydromorphone utilising morphinone reductase has also been described in U.S. Pat. No. 5,571,685. In addition, the catalytic rearrangement of morphine to hydromorphone in dichloromethane using a complex rhodium based catalyst pre-activated with hydrogen has been described in U.S. Pat. No. 5,847,142. A similar process involving the catalytic rearrangement of morphine to hydromorphone in dilute hydrochloric acid using palladium (Pd) or platinum (Pt) catalysts pre-activated by hydrogen is also known (see U.S. Pat. No. 6,589,960). In both of these catalytic processes, morphine is rearranged directly to hydromorphone eliminating the need for two separate reaction steps. However, the yields obtained by these processes are generally relatively low.

A process for the preparation of 8,14-dihydrothebaine from thebaine has also previously been described in U.S. Pat. No. 3,812,132. This process involves heating the thebaine with a thermally decomposable hydrazide of a sulphonic acid such as a benzenesulphonic acid to generate diimine for effecting the reduction of thebaine to 8,14-dihydrothebaine. Other compounds described as being useful for generating diimine include alkyl sulphonic acid hydrazides or when a proton supplying substance such as an alcohol or acid is present, disodium azodicarboxylate. The 8,14-dihydrothebaine generated was then hydrolysed to dihydrocodeinone.

Oripavine is an alkaloid believed to be an intermediate in the conversion of thebaine to morphine in P. somniferum (Parker, H. I., 1972; Brockmann-Hanssen, E., 1984). Morphine itself is a valuable narcotic alkaloid and finds many applications in medical therapies and treatments. Hence, the use of morphine in the synthesis of hydromophone is undesirable.

### SUMMARY OF THE INVENTION

The present invention stems from the recognition that the 8,14-double bond of oripavine may be selectively reduced by diimine to generate an intermediate which may then be readily converted to hydromorphone or a physiologically acceptable salt thereof.

Accordingly, in a first aspect of the present invention there is provided a method for preparing hydromorphone or a physiologically acceptable salt thereof, the method comprising:
reacting a compound of formula I with diimine in a reaction mixture to produce a compound of formula II; and
converting the compound of formula II to hydromorphone or physiologically acceptable salt thereof;
wherein the compounds of formula I and II are as follows: 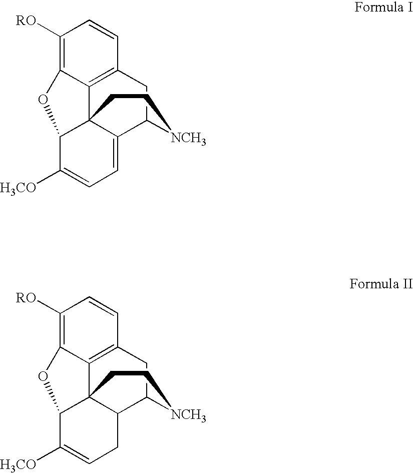
and R is hydrogen or a protecting group.

In addition to separate addition of the diimine to the reaction mixture, any suitable process in which diimine is generated in the reaction mixture may be utilised for producing the compound of formula II. Typically, the diimine will be generated by the decomposition of an azo compound in situ in the presence of the compound of formula I. Preferably, the azo compound will be selected from hydrazine, azodicarboxylates and hydrazides and most preferably, will be an azodicarboxylate or a hydrazide. The decomposition of the diimine generating compound will normally comprise thermal decomposition of the compound. As such, a method embodied by the invention may involve applying heat to the reaction mixture to promote the thermal decomposition of the azo compound.

More generally, the method will preferably comprise generating the diimine in the presence of the compound of formula I under conditions such that the compound of formula I reacts with the diimine to produce the compound of formula II.

When an acid is produced by the decomposition of the azo compound, the reaction of the compound of formula I with the diimine will preferably be conducted in the presence of a base for neutralising the acid to reduce or eliminate the possibility of the acid undergoing further reaction to generate product(s) that reduce the yield of diimine or otherwise impact on the yield of the compound of formula II.

The compound of formula II may be isolated and subsequently converted to hydromorphone, or be converted to hydromorphone without first being isolated. In the former instance, the compound may be crystallised and the crystallised compound utilised in the conversion process to hydromorphone.

In a preferred embodiment the compound of formula II is isolated as a solid then converted to the hydromorphone or physiologically acceptable salt thereof in a suitable solvent. In a further preferred embodiment the physiologically acceptable salt is hydromorphone hydrochloride. In yet another preferred embodiment the compound of formula II is 8,14-dihydrooripavine.

In another preferred embodiment the compound of formula II is separated from the reaction mixture then converted to the hydromorphone or physiologically acceptable salt thereof in a suitable solvent. In a further preferred embodiment the physiologically acceptable salt is hydromorphone hydrochloride. In yet another preferred embodiment the compound of formula II is 8,14-dihydrooripavine.

Preferably, the compound of formula II will be converted to hydromorphone by hydrolysis and typically, by subjecting the compound to acid hydrolysis. As a result of the production of hydrochloric acid, the pH in the stomach is relatively low and may therefore, facilitate the hydrolysis of the compound of formula II in vivo.

Accordingly, in another aspect of the present invention there is provided a method for treating a mammal for pain, the method comprising:
administering to the mammal an effective amount of a compound of formula II or physiologically acceptable salt thereof which is converted to hydromorphone or physiologically acceptable salt thereof in the mammal;
wherein the compound of formula II is as follows: 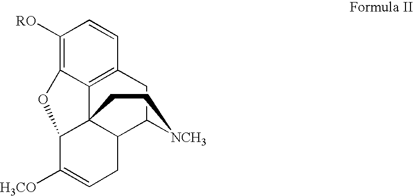
and R is hydrogen or a physiologically acceptable protecting group.

Still further, in another aspect of the present invention there is provided the use of a compound of formula II or physiologically acceptable salt thereof in the manufacture of a medicament for treatment of a mammal for pain with conversion of the compound to hydromorphone or physiologically acceptable salt thereof in the mammal, wherein the compound of formula II is as follows: 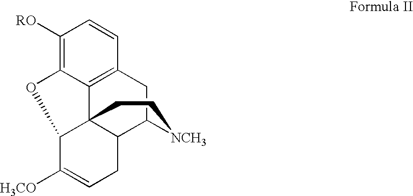
and R is hydrogen or a physiologically acceptable protecting group.

Typically, the R group of a compound of formula I will be hydrogen. Hence, in this instance the compound of formula I will thereby be oripavine and the compound of formula II will be 8,14-dihydrooripavine. The compound 8,14-dihydrooripavine is therefore a key compound in the preparation of hydromorphone from oripavine in accordance with a preferred embodiment of a method of the invention.

Hence, in another aspect of the present invention there is provided a method for preparing 8,14-dihydrooripavine or a physiologically acceptable salt thereof, the method comprising:
reacting oripavine with diimine in a reaction mixture to produce 8,14-dihydrooripavine; and
isolating the 8,14-dihydrooripavine or physiologically acceptable salt thereof.

In a further aspect of the present invention there is provided a method for treating a mammal for pain, the method comprising administering to the mammal an effective amount of 8,14-dihydrooripavine or physiologically acceptable salt thereof.

Being an alkaloid, 8,14-dihydrooripavine itself may also have physiological activity. Hence in still another aspect the present invention relates to a method for treating a disease or condition in a mammal responsive to 8,14-dihydrooripavine, comprising administering to the mammal an effective amount of 8,14-dihydrooripavine or a physiologically acceptable salt thereof to the mammal.

In yet another aspect there is provided hydromorphone or a physiologically acceptable salt thereof prepared by a method of the invention.

In still another aspect there is provided 8,14-dihydrooripavine or physiologically acceptable salt thereof prepared by a method of the invention.

In a further aspect of the present invention there is provided 8,14-dihydrooripavine or a physiologically acceptable salt thereof.

In yet another aspect of the present invention there is provided a pharmaceutical composition comprising 8,14-dihydrooripavine or a physiologically acceptable salt thereof together with a pharmaceutically acceptable carrier.

In a still further aspect of the present invention there is provided the use of 8,14-dihydrooripavine or a physiologically acceptable salt thereof in the manufacture of a medicament for treating a mammal for pain.

The mammal to which a compound is administered in accordance with the invention may for instance be a primate, a rabbit, a rodent such as a mouse, or any mammal responsive to the compound. Preferably, the mammal will be a human being.

### DETAILED DESCRIPTION OF THE INVENTION

Various processes employing diimine for selectively reducing carbon-carbon double bonds are known in the art and any appropriate processes may be utilised in the method of the present invention, such as the Eppenberger process (Eppenberger et al; 1968) which utilises air or oxygen to oxidise hydrazine to generate diimine, or processes that start from hydroxylamines, for instance hydroxylamine-O-sulphonic acid in alkaline solution, or the thermal degradation of anthracene-9, 10-biimine. However, due to drawbacks of these processes and in particular the combustibility of hydrazine in combination with oxygen, it is preferable to achieve the generation of diimine by the thermal decomposition of a suitable azo compound. Suitable such processes are described in U.S. Pat. No. 3,812,132 the contents of which are incorporated herein in its entirety. Preferred azo compounds, include but are not limited to, substituted or unsubstituted aryl sulphonic acid hydrazides, substituted or unsubstituted alkyl sulphonic acid hydrazides, substituted or unsubstituted aralkyl sulphonic acid hydrazides, substituted or unsubstituted acyl hydrazides, azodicarboxylate and salts thereof, and substituted or unsubstituted heterocyclic or carbocyclic sulphonic acid hydrazides.

An aryl sulphonic acid hydrazide utilised in a method of the invention may have an aryl group selected from substituted or unsubstituted single ring systems or polycyclic groups, which may include one or more heteroatoms typically selected from N, S and O. Typically the aryl group will have a ring with 5 or 6 ring members. The aryl group may for instance be selected from phenyl, bi-phenyl, alkylphenyls such as C₁-C₄alkylphenyl, and polyalkylphenyls. 2,4,6-triisopropylbenzene sulphonyl hydrazide and p-toluenesulphonyl hydrazide are particularly preferred such azo compounds.

An alkyl sulphonic acid hydrazide may have an alkyl group selected from straight and branched chain alkyl groups. Straight or branched chain alkyl groups with a C₁-C₁₂ carbon backbone are preferred and most preferably, lower alkyl groups. The term “lower alkyl” group is to be taken to mean a C₁-C₆ alkyl.

Aralkyl groups are alkyl groups substituted with at least one aryl group such as an aryl group described above. Preferably, an aralkyl sulphonic acid hydrazide utilised will have a straight or branched alkyl group with a C₁-C₁₂ carbon backbone and most preferably, a C₁-C₆ carbon backbone.

When an azodicarboxylate or salt thereof is utilised in a method of the invention, the reaction will typically take place in the presence of one or more additional reagents that donate or generate H⁺ or H₃O⁺ ions such as an acid, alcohol, or water, for enabling the generation of the diimine. Preferred azodicarboxylate salts include cations such as sodium or potassium.

Preferred acylhydrazines include haloacylhydrazines such as chloracetyl hydrazide.

Heterocyclic groups are cyclic carbon ring systems incorporating one or more heteroatoms selected, from N, S and O. Preferred heterocyclic or carbocyclic sulphonic acid hydrazides have one or more heterocyclic or carbocyclic rings typically with five or six ring members and zero or one or more multiple bonds, for instance such as 8-quinolinesulphonyl hydrazide or 2-thiophenesulphonyl hydrazide.

Substituent groups, if any, should preferably not react with any of the reagents utilised in the reaction mixture or produced during a reaction embodied by the present invention. In particular, in considering azo compounds for use in a method of the invention, one criteria for selection of the azo compound is that by-products resulting from the oxidation of the compound in the generation of diimine should desirably be substantially inert with respect to diimine or the alkaloid of formula I and the resulting intermediate of formula II under the reaction conditions utilised.

The solvent selected for the reaction will also be substantially inert with respect to the reagents or products generated in a method of the invention. The solvent will normally have a boiling point which facilitates the thermal decomposition of the selected azo compound. Preferably, the solvent will have a boiling point of at least 65° C. and most preferably, about 80° C. or higher. Suitable solvents may be for instance water, C₁-C₅ alcohols, morpholine, diethyl carbonate, toluene, methyl oxitol, diglyme, and ethanolamine.

For reactions in organic solvents it is not necessary to add a base, however, the use of a base is preferred as acidic by-products may be generated from alkyl and aryl sulphonic acid hydrazides, for instance, and impact on the yield of the intermediate of formula II or further react to form sulphur compounds (e.g., S-phenyl benzenethiosulphonate and diphenyl sulphide when benzenesulphonic acid hydrazides are utilised). These may also impact on the yield of diimine generated (see U.S. Pat. No. 3,812,132).

In water based reactions a molar excess of strong base should be present to dissolve the oripavine and ensure a high yield.

Suitable bases include organic and inorganic compounds, such as for instance hydroxides, carbonates, bicarbonates, or amines.

The preferred solvent system is water with a minor amount of alcohol co-solvent and a base. However, morpholine is also very effective as it may be utilised alone as both a solvent and base.

The amount of azo compound required for achieving the reduction of the 8,14-double carbon bond of oripavine for maximum yield of the compound of formula II will depend on the azo compound utilised and may be determined by routine experimentation. That is, the reaction of the invention may be conducted and the yield of the compound of formula II determined before repeating the reaction utilising a greater or lesser amount of the azo compound. Preferably, the azo compound will be in molar excess to the alkaloid compound of formula I and typically, in an excess of about 6 times or less and most usually, in molar excess of between about 2 and 4 times.

The protecting group may be any suitable group for protecting the hydroxy group of a phenol compound under the reaction conditions used in a reaction embodied by the present invention. Preferably, the conversion of the compound of formula II to hydromorphone will be achieved in a single reaction step and the protecting group will be one which is removed under the conditions utilised in this step. When the compound is converted to hydromorphone or a physiologically acceptable salt thereof by acid hydrolysis, the protecting group may for instance be selected from those which form ketals, esters and ethers with the phenol oxygen of oripavine and which can be removed in the presence of hydronium ion. The protecting group may for instance be selected from methoxymethyl, benzyl, isopropyl, cyclohexyl, t-butyl, tetrahydropyranyl, phenacyl, cyclopropylmethyl, trimethylsilyl, acetyl, propanoyl, pivaloyl, and benzoyl. Typically, the protecting group will be other than methyl and more generally, other than an alkyl group.

When administered to a mammal, the protecting group of the compound of formula II will desirably be essentially non-toxic to the mammal when removed from the compound in vivo, or otherwise have minimal physiological effect on the mammal.

Preferably, oripavine will typically be utilised without protecting group modification of the phenol hydroxy of the alkaloid in the synthesis of hydromorphone or physiologically acceptable salt thereof. The conversion of oripavine to hydromorphone hydrochloride is illustrated in Scheme 1.

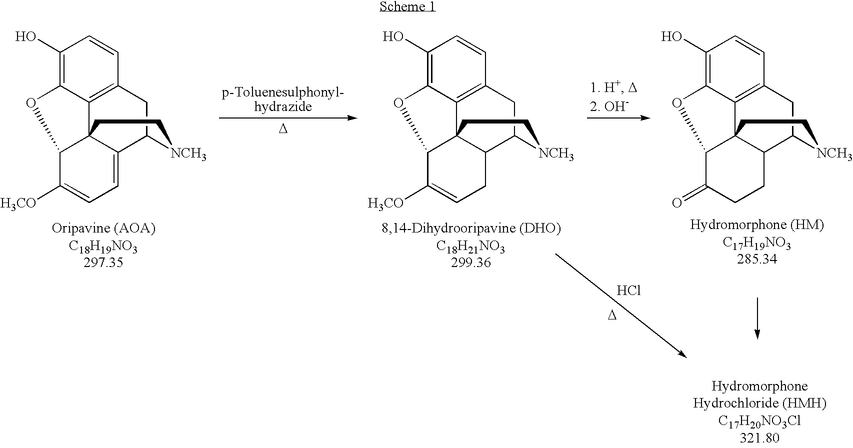

The hydrolysis may be promoted by any suitably acidic conditions, for instance in water or lower alcohols, by the addition of mineral or organic acids. In a preferred embodiment, hydrochloric acid is used to directly generate the physiologically active salt, hydromorphone hydrochloride.

The 8,14-dihydrooripavine may be separated from the reaction mixture then converted to hydromorphone or physiologically acceptable salt thereof in a suitable solvent. In an alternative embodiment the 8,14-dihydrooripavine is isolated as a solid prior to conversion to hydromorphone or physiologically acceptable salt thereof. When isolated as a solid, the 8,14-dihydrooripavine may be re-crystallised or otherwise purified before subsequent use. When the 8,14-dihydrooripavine is separated from the reaction mixture, it will normally be extracted from the reaction mixture utilising a suitable organic solvent. The 8,14-dihydrooripavine may then be back extracted into another solvent, typically an acidic aqueous solution, in which the 8,14-dihydrooripavine is converted to hydromorphone. The compound of formula II or 8,14-dihydrooripavine, or physiologically acceptable salt thereof, may be formulated into a pharmaceutical composition incorporating a pharmaceutically acceptable carrier for the purpose of administration.

Pharmaceutically acceptable salts include carboxylate salts (e.g., C₁₋₈ alkyl, C₃₋₈ cycloalkyl, aryl, C₂₋₁₀ heteroaryl, or C₂₋₁₀ non-aromatic heterocyclic) and others that are within a reasonable benefit/risk ratio, pharmacologically effective and suitable for contact with the tissues of patients without undue toxicity, irritation, or allergic response. Representative salts include hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, and laurylsulfonate. These may include alkali metal and alkali earth cations such as sodium, potassium, calcium, and magnesium, as well as non-toxic ammonium, quaternary ammonium, and amine cations such as tetramethyl ammonium, methylamine, trimethylamine, and ethylamine. See for example, S. M. Berge, et al., “Pharmaceutical Salts,” J. Pharm. Sci., 1977, 66:1-19, which is incorporated herein by reference.

Pharmaceutical compositions include sterile solutions which may for instance, be prepared by incorporating the desired amount of the compound in the selected liquid carrier prior to sterilising the solution by filtration.

For oral administration, the selected active compound may be formulated into any orally acceptable carrier deemed suitable. In particular, the compound may be formulated with an inert diluent, an assimilable edible carrier or it may be enclosed in a hard or soft shell gelatin capsule. More particularly, the composition may be provided in the form of ingestible tablets, buccal tablets, troches, capsules, elixires, suspensions or syrups.

A pharmaceutical composition of the invention may also incorporate one or more preservatives such as parabens, chlorobutanol, phenol, sorbic acid, and thimersal. In addition slow release formulations, such as for the release of the compound in the stomach, are expressly encompassed by the present invention. Any appropriate such formulations known in the art may be utilised. Preferred such compositions are ones which release the compound in the gastrointestinal tract over a period of up to about 12 hours and most preferably, between about one and four hours. The delayed release of the active compound may be achieved by the use of agents such as, for example, aluminium monosterate and gelatin.

Tablets, troches, pills, capsules and the like may also contain one or more of the following: a binder such as gum tragacanth, acacia, corn starch or gelatin; a disintegrating agent such as corn starch, potato starch or alginic acid; a lubricant such as magnesium sterate; a sweetening agent such as sucrose, lactose or saccharin; and a flavouring agent.

Pharmaceutically acceptable carriers include any suitable conventionally known solvents, dispersion media and isotonic preparations or solutions. Use of such ingredients and media for pharmaceutically active substances is well known. Except insofar as any conventional media or agent is incompatible with 8,14-dihydrooripavine or a compound of formula II, or physiologically acceptable salt thereof, use of the media or agent is included. Supplementary physiologically active ingredients can also be incorporated in a pharmaceutical composition of the invention if desired.

In addition, the pharmaceutical compositions may be formulated in dosage unit form for ease of administration and uniformity of dosage. Dosage unit form as used herein is to be taken to mean physically discrete units suited as unitary dosages for the subject to be treated, each unit containing a predetermined quantity of the selected active compound calculated to produce the desired physiological effect in association with the relevant carrier used. As will be appreciated, the amount of the selected compound utilised in the composition will be such that a suitable effective dosage will be delivered to the subject taking into account the proposed mode of administration.

The dosage of the selected compound administered will depend on a number of factors including whether the agent is to be administered for prophylactic or therapeutic use, the disease or condition for which the agent is intended to be administered, the severity of the disease or condition, the age of the subject, and related factors including weight and general health of the subject, as may be determined by the physician or attendant in accordance with accepted principles. For instance, a low dosage may initially be given which is subsequently increased at each administration following evaluation of the subjects response. Similarly, frequency of administration may be determined in the same way, that is, by continuously monitoring the subject's response between each dosage and if necessary, increasing the frequency of administration or alternatively, reducing the frequency of administration.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed anywhere before the priority date of each claim of this application.

In order that the nature of the present invention may be more clearly understood, preferred forms thereof will now be described with reference to the following non-limiting examples.

### EXAMPLE 1

Synthesis of 8,14-dihydrooripavine in Morpholine

To oripavine (51.3 g) in morpholine (120 ml) at 126° C. was added over 1 hour a solution of p-toluenesulphonyl hydrazide (64.6 g) in morpholine (120 ml). The solution was heated at 125-128° C. for a further 40 minutes then cooled to 90° C. and warm water (250 mL at 50° C.) added. The mixture was allowed to cool to 70° C. then further water (250 ml) was added. The slurry was cooled to 0-5° C. and stirred for 20 minutes, and the pH then adjusted by the addition of concentrated phosphoric acid to pH 9.2. The final slurry was stirred for 20 minutes then the 8,14-dihydrooripavine (40.6 g dry weight, 81% yield) isolated by filtration and dried under vacuum.

### EXAMPLE 2

Purification of 8,14-dihydrooripavine by Recrystallisation

A portion of the 8,14-dihydrooripavine (35.7 g) from Example 1 was dissolved in ethanol (750 ml) at reflux. Activated carbon (2.0 g) was added and the mixture was stirred for 5-10 minutes before filtering with an ethanol rinse (100 ml). The filtrate was concentrated in vacuo to remove most of the ethanol (570 ml) then cooled to 0-5° C. and aged for 30 minutes. Filtration of the slurry and vacuum drying provided recrystallised 8,14-dihydrooripavine (32.5 g). This was recrystallised once more from ethanol, as above but omitting the carbon treatment and cooling only to 20-25° C., to provide a purified 8,14-dihydrooripavine (25.6 g, 72%) for spectral characterisation. All data was consistent with the proposed structure. The m.p. was 230-231° C. (uncorrected).

¹H-NMR (CDCI₃, 400 MHz): δ 1.51-2.37 (m, 5H, 2×H₁₅, H₁₄, 2×H₈), 2.43 s, 3H, NCH₃), 2.48-3.32 (m, 5H, 2×H₁₀, 2×H₁₆, H₉), 2.40 (OCH₃), 4.71 (d, J=6.5 Hz, 1H, H₇), 4.77 (s, 1H, H₅), 6.55 (d, J=8.2 Hz, 1H, H₁), 6.67 (d, J=8.2 Hz, 1H, H₂).

¹³C-NMR (CDCI₃, 100 MHz): δ 20.6 (C₁₀), 23.6 (C₈), 35.1 (C₁₅), 39.0 (C₁₄), 42.48 (NCH₃), 42.53 (C₁₃), 46.6 (C₁₆), 54.4 (OCH₃), 59.0 (Cg), 88.5 (C₅), 98.4 (C₇), 117.5 (C₂), 119.2 (C₁) 125.0 (C₁₁), 128.7(C₁₂), 139.9(C₃), 143.9(C₄), 152.1 (C₆).

EI MS: m/z=299.1515 (M+, C₁₈H₂₁NO₃).

FTIR (KBr): 2910, 2845, 1661 (C₆═C₇) cm⁻¹.

### EXAMPLE 3

Synthesis of 8,14-dihydrooripavine in Water with N-Butanol

To oripavine (20.37 g) in water (37 ml) with sodium hydroxide (3.37 g) at 97-100° C. was added over 3.5 hours a solution of p-toluenesulphonyl hydrazide (30.85 g) in a mixture of water (133 ml) and n-butanol (14 ml) with sodium hydroxide (7.71 g). The solution was heated at reflux for a further 45 minutes then cooled to 40° C. The pH was adjusted to 9.1 by the addition of 56% (v/v) acetic acid. The resultant slurry was stirred for 45 minutes at 40° C. then the 8,14-dihydrooripavine was isolated with water (60 ml) and ethanol washes (40 ml) and vacuum dried (18.10 g dry, 90% yield).

### EXAMPLE 4

Purification of 8,14-dihydrooripavine in an Aqueous Mixture

8,14-Dihydroorpavine (25 g) was dissolved in a mixture of water (70 ml), acetonitrile (75 ml), ethanol (17.5 ml) and acetic acid (56% v/v, 13.5 ml) with sodium metabisulphite (0.5 g) at 45-50° C. The solution was pumped through an encapsulated carbon disk (47 mm diameter, KB-B carbon) at 12.5 ml/min. The disk was rinsed with a mixture of acetonitrile:ethanol:water (0.5:0.1:0.4, 25 ml). The solution was filtered through a 0.7 μm fiberglass disk. The pH of the filtrate was adjusted to 9.1 with 28% w/v ammonium hydroxide and the resulting slurry was cooled to 3° C. The purified 8,14-dihydrooripavine was isolated by filtration with a water rinse (50 ml) and vacuum dried (21.5 g dry, 87% yield).

### EXAMPLE 5

Synthesis of Hydromorphone Base Directly via 8,14-dihydrooripavine

To oripavine (50.9 g) in morpholine (120 ml) at 126-128° C. was added over 65 minutes a solution of p-toluenesulphonyl hydrazide (64.6 g) in morpholine (120 ml). The solution was heated at 126-128° C. for a further 40 minutes then cooled to 90-100° C. A mixture of 30:70 n-butanol:toluene (300 ml) was added followed by water (200 ml). The butanol:toluene phase was separated and the water extracted twice more with butanol:toluene (2×100 ml). The butanol:toluene extracts were combined and washed with water (100 ml) which was back-extracted with butanol:toluene (30 ml).

The combined butanol:toluene solution was then extracted twice with 2M aqueous HCl (200 ml+50 ml) to provide an aqueous acid solution of 8,14-dihydrooripavine.

The acid solution was heated at 70-85° C. for 2 hours then cooled to 55-65° C. Activated carbon (1.9 g) was added and the solution filtered after 10 minutes with a water (50 ml) rinse. The carbon treatment was repeated twice more, but with smaller water rinses (10 ml). The pH was then adjusted to pH 9.1 by the addition of 40% w/v potassium hydroxide solution. The resultant slurry was filtered and the hydromorphone base (28.0 g dry weight, 57% yield) vacuum dried.

### EXAMPLE 6

Purification of Hydromorphone Base

A portion of the hydromorphone base (26.0 g) from Example 5 was dissolved in ethanol (910 ml) at reflux. The majority of the ethanol (750 ml) was removed by concentration in vacuo and the slurry cooled to 0-5° C. Filtration provided a solid which was recrystallised a second time, as above, to provide a purified hydromorphone base (21.2 g, 81%). The identity of the solid was confirmed by spectral characterisation. The m.p. was 264-266° C. (lit. 262.5-263° C.).

¹H-NMR (DMSO, 400 MHz): δ 0.92-2.18 (m, 7H, 2×H₁₅, H₁₄, 2×H₇, 2×H₈), 2.25 (s, 3H, NCH₃), 2.34-3.04 (m, 5H, 2×H₁₀, 2×H₁₆, Hg), 4.78 (s, 1H, H₅), 6.47 (d, J=8 Hz, 1H, H₁), 6.52 (d, J=8 Hz, 1H, H₂), 9.15 (S, 1H, OH).

¹³C-NMR (DMSO, 100 MHz); δ 19.6 (C₁₀), 25.1 (C₈), 34.9 (C ₁₅), 38.9-40.2 (C₇, overlapping with DMSO signals), 41.5 (₁₄), 42.6 (NCH₃), 46.3, (C₁₆), 46.4 (C₁₃), 58.4 (C₉), 90.5 (C₅), 117.0 (C₂), 119.3 (C₁), 124.6 (C₁₁), 127.5 (C₁₂), 139.3 (C₃), 144.0 (C₄), 209.0 (C₆).

El MS: m/z=285.1365 (M+, C₁₇H₁₉NO₃).

FTIR (KBr): 1729 (C═O) cm⁻¹

### EXAMPLE 7

Direct Synthesis of Hydromorphone Hydrochloride from 8,14-dihydrooripavine

8,14-Dihydrooripavine (5.00 g) which had been recrystallised once from ethanol was heated in a mixture of water (2.5 ml) and 32% w/w hydrochloric acid (2.5 ml) under nitrogen for 50 minutes at 75° C. The resulting slurry was cooled to 60° C. and warm ethanol (20 ml at 60° C.) added slowly. The mixture was cooled and aged at 0-5° C. prior to isolation of the product by filtration with ethanol rinses. Vacuum drying provided a colourless hydromorphone hydrochloride (5.00 g, 93% yield).

### EXAMPLE 8

Direct Synthesis of Hydromorphone Hydrochloride from 8,14-dihydrooripavine

Purified 8,14-Dihydrooripavine (120 g) was heated in a mixture of water (96 ml), ethanol (120 ml) and 32% w/w hydrochloric acid (48 ml) under reflux (83° C.) for 70 minutes. One quarter of this solution was then separated and treated further as below.

To the solution portion from above at 50-60° C. was added methanol (30 ml). The solution was then cooled to 5° C. and aged for 6 hours. Further ethanol (60 ml) was then added over 6 hours. The suspension was aged for 6 hours at 5° C. then the product was isolated with an ethanol rinse (45 ml) and vacuum dried to give colourless hydromorphone hydrochloride (26.5 g, 82% yield). The identity and purity of this material was confirmed by HPLC analysis. The assay was 100.1% w/w. No impurities were detected above 0.1% by relative peak area.

### EXAMPLE 9

Synthesis of Hydromorphone Hydrochloride from 8,14-dihydrooripavine via Hydromorphone Base

8,14-Dihydrooripavine (8.06 g) was heated in a mixture of water (13 ml) and concentrated hydrochloric acid (3 ml) under nitrogen for 50 minutes at 75° C. The solution was then cooled to 50-60° C. Activated carbon (0.56 g) and celite (0.15 g) were added and the solution was filtered after 10 minutes, with water rinses (2×5 ml). n-Butanol (2.5 ml) was added and the pH was adjusted to 9.1 by the addition of 20% w/v sodium hydroxide. The slurry was cooled to 20° C. and aged for 1 hour then filtered with a water (10 ml) rinse. Vacuum drying provided the hydromorphone base (7.13 g, 91% yield).

An amount of the hydromorphone base (6.00 g) was dissolved by heating in a mixture of water (4.2 ml) and concentrated hydrochloric acid (2.8 ml) at 70° C. The solution was then cooled to 60-65° C. and warm ethanol (24 ml at 60-65° C.) added slowly. The mixture was cooled and aged at 0-5° C. then the product isolated by filtration with ethanol rinses. Vacuum drying provided the hydromorphone hydrochloride (5.92 g, 89% yield from hydromorphone base).

### EXAMPLE 10

HPLC Analysis of 8,14-dihydrooripavine and Hydromorphone Compositions

Hydromorphone compositions were assayed and impurities quantified using a gradient reverse phase high pressure liquid chromatography (HPLC) method. The eluant was an acetonitrile water mixture at alkaline pH. The alkaline pH provided an improved resolution of impurities versus the previously published pharmacopeial methods, which have used acidic mobile phases. The method involved injecting the aqueous solution of hydromorphone hydrochloride onto a Phenomenex Gemini C18 column (250×4.6×5 μm) with a flow rate of 1 ml/min and UV detection at 284 nm. The gradient is shown below. Mobile phase A included ammonium bicarbonate (7.9 g) and sodium metabisulphite (1.2 g) in purified water (1 L) adjusted to pH 9.6 with concentrated ammonium hydroxide. Mobile phase B was acetonitrile. Mobile phase C was purified water.

| | | | | Rate of |
|---|---|---|---|---|
| Time (min) | Line A | Line B | Line C | Change |
| 0 | 10% | 2% | 88% | — |
| 25 | 10% | 30% | 60% | linear |
| 35 | 10% | 30% | 60% | linear |
| 40 | 10% | 2% | 88% | — |

For the analysis of 8,14-dihydrooripavine a similar alkaline HPLC system was used, but with a Waters XTerra column and the gradient commencing from 20% acetonitrile and changing to 53% over 25 minutes. The use of other HPLC columns and gradient conditions may provide further optimised separations under alkaline conditions for the alkaloids described herein.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for preparing hydromorphone or a physiologically acceptable salt thereof, the method comprising:
reacting a compound of formula I with diimine in a reaction mixture to produce a compound of formula II; and
converting the compound of formula II to hydromorphone or a physiologically acceptable salt thereof;
wherein the compounds of formula I and II are as follows: 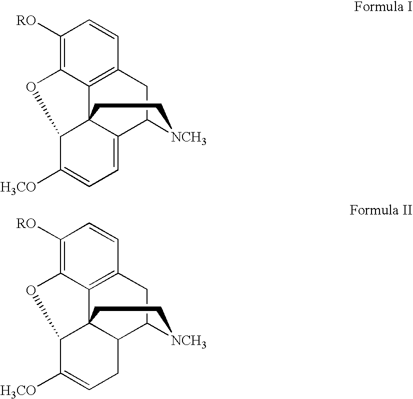
and R is hydrogen or a protecting group.

2. A method according to claim 1, wherein R is hydrogen.

3. A method according to claim 1 comprising generating the diimine in the reaction mixture under conditions such that the compound of formula I reacts with the diimine to produce the compound of formula II.

4. A method according to claim 3, wherein the diimine is generated by the decomposition of an azo compound in the reaction mixture.

5. A method according to claim 4, wherein the decomposition is thermal decomposition of the azo compound and the method further comprises applying heat to the reaction mixture to promote the thermal decomposition of the azo compound.

6. A method according to claim 4, wherein the azo compound is selected from the group consisting of hydrazine, azodicarboxylates and hydrazides.

7. A method according to claim 4, wherein the azo compound is selected from the group consisting of substituted or unsubstituted aryl sulfonic acid hydrazides, substituted or unsubstituted alkyl sulfonic acid hydrazides, substituted or unsubstituted aralkyl sulfonic acid hydrazides, substituted or unsubstituted acyl hydrazides, azodicarboxylates and salts thereof, and substituted or unsubstituted heterocyclic or carbocyclic sulphonic acid hydrazides.

8. A method according to claim 7, wherein the azo compound is an aryl sulfonic acid hydrazide with an aryl group consisting of a substituted or unsubstituted single ring system or polycyclic group.

9. A method according to claim 8, wherein the azo compound is 2,4,6-triisopropylbenzene sulphonyl hydrazide or p-toluenesulphonyl hydrazide.

10. A method according to claim 8, wherein the aryl group incorporates one or more heteroatoms selected from N, S and O.

11. A method according to claim 8, wherein the aryl group is selected from the group consisting of phenyl, bi-phenyl, alkylphenyls and polyalkylphenyls.

12. A method according to claim 11, wherein the alkylphenyl is C₁-C₄ alkylphenyl.

13. A method according to claim 7, wherein the azo compound is an alkyl sulfonic acid hydrazine having straight or branched chain alkyl group with a C₁-C₁₂ carbon backbone.

14. A method according to claim 13, wherein the carbon backbone is C₁-C₆.

15. A method according to claim 7, wherein the azo compound is an aralkyl sulphonic acid hydrazide having an aralkyl group with a straight or branched chain alkyl having a C₁-C₁₂ carbon backbone.

16. A method according to claim 15, wherein the carbon backbone is C₁-C₆.

17. A method according to claim 7, wherein the azo compound is an azodicarboxylate or salt thereof.

18. A method according to claim 17, wherein the azodicarboxylate is utilised in the presence of a proton donor for donating a proton for generation of the diimine.

19. A method according to claim 18, wherein the proton donor is selected from the group consisting of water, alcohols, and acids.

20. A method according to claim 7, wherein the azo compound is a haloacylhydrazine.

21. A method according to claim 20, wherein the haloacylhydrazine is chloroacetyl hydrazine.

22. A method according to claim 7, wherein the azo compound is a heterocyclic or carbocyclic sulphonic acid hydrazide of one or more ring members.

23. A method according to claim 22, wherein the ring member is a five or six membered ring having zero or more multiple bonds.

24. A method according to claim 22, wherein the azo compound is 8-quinolinesulphonyl hydrazine or 2-thiophenesulphonyl hydrazide.

25. A method according to claim 1, wherein the reaction mixture has an alkaline pH.

26. A method according to claim 1, wherein the compound of formula II is isolated as a solid then converted to hydromorphone or physiologically acceptable salt thereof in a suitable solvent.

27. A method according to claim 26, wherein the physiologically acceptable salt is hydromorphone hydrochloride.

28. A method according to claim 26, wherein the compound of formula II is 8,14-dihydrooripavine.

29. A method according to claim 26, wherein the compound of formula II is 8,14-dihydrooripavine and the physiologically acceptable salt is hydromorphone hydrochloride.

30. A method according to claim 1 wherein in the compound of formula II is separated from the reaction mixture then converted to hydromorphone or physiologically acceptable salt thereof in a suitable solvent.

31. A method according to claim 30, wherein the physiologically acceptable salt is hydromorphone hydrochloride.

32. A method according to claim 30, wherein the compound of formula II is 8,14-dihydrooripavine.

33. A method according to claim 30, wherein the compound of formula II is 8,14-dihydrooripavine and the physiologically acceptable salt is hydromorphone hydrochloride.

34. A method for preparing 8,14-dihydrooripavine or a physiologically acceptable salt thereof, the method comprising:
reacting oripavine with diimine in a reaction mixture to produce 8,14-dihydrooripavine; and
isolating the 8,14-dihydrooripavine or physiologically acceptable salt.

35. A method according to claim 34, comprising generating the diimine in the reaction mixture under conditions such that the oripavine reacts with the diimine to produce the 8,14-dihydrooripavine.

36. A method according to claim 35, wherein the diimine is generated by the decomposition of an azo compound in the reaction mixture.

37. A method according to claim 36, wherein the decomposition is thermal decomposition of the azo compound and the method further comprises applying heat to the reaction mixture to promote the thermal decomposition of the azo compound.

38. A method according to claim 36, wherein the azo compound is selected from the group consisting of substituted or unsubstituted aryl sulfonic acid hydrazides, substituted or unsubstituted alkyl sulfonic acid hydrazides, substituted or unsubstituted aralkyl sulfonic acid hydrazides, substituted or unsubstituted acyl hydrazides, azodicarboxylates and salts thereof, and substituted or unsubstituted heterocyclic or carbocyclic sulphonic acid hydrazides.

39. A method for treating a mammal for pain, the method comprising:
administering to the mammal an effective amount of a compound of formula II or a physiologically acceptable salt thereof, which is converted to hydromorphone or physiologically acceptable salt thereof in the mammal;
wherein the compound of formula II is as follows: 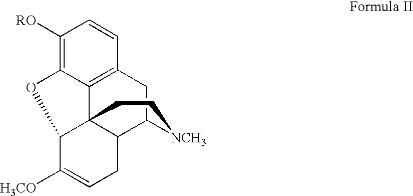
and R is hydrogen or a physiologically acceptable protecting group.

40. A method according to claim 39, wherein R is hydrogen.

41. A method according to claim 39, wherein the compound of formula II is converted to the hydromorphone or physiologically acceptable salt thereof in the stomach of the mammal.

42. A method according to claim 39, wherein the compound of formula II is administered orally to the mammal.

43. A method according to claim 39, wherein the compound of formula II is administered in a slow release formulation.

44. A method for treating a mammal for pain comprising administering to the mammal an effective amount of 8,14-dihydrooripavine or a physiologically acceptable salt thereof.

45. A method according to claim 44 wherein the mammal is a human being.

46. 8,14-dihydrooripavine or a physiologically acceptable salt thereof.

47. A pharmaceutical composition comprising 8,14-dihydrooripavine or a physiologically acceptable salt thereof together with a pharmaceutically acceptable carrier.
